(19) United States
(12) Patent Application Publication
(10) Pub. No.: US 2021/0145315 A1
(43) Pub. Date: May 20, 2021

(54) FOOT PRESSURE MEASUREMENT APPARATUS, METHOD, AND NON-TRANSITORY TANGIBLE MACHINE-READABLE MEDIUM THEREOF

(71) Applicant: Institute For Information Industry, Taipei (TW)

(72) Inventors: Yan-Tong LIU, Taipei (TW); Chih-Hao HSU, Taipei (TW)

(21) Appl. No.: 16/687,085

(22) Filed: Nov. 18, 2019

(51) Int. Cl. A61B 5/103; G06N 3/12; G01L 5/16; A61B 5/00

(52) U.S. Cl. CPC A61B 5/1036; A61B 5/7264; G01L 5/16; G06N 3/126

(57) ABSTRACT

A foot pressure measurement apparatus, method, and non-transitory tangible machine-readable medium thereof are provided. The foot pressure measurement apparatus stores multiple first pressure value sets corresponding to a first position set of a foot and multiple second pressure value sets corresponding to a second position set of the foot. A first position quantity of the first position set is smaller than a second position quantity of the second position set. The foot pressure measurement apparatus decides multiple candidate weight set. For each candidate set, the foot pressure measurement apparatus calculates multiple first center of pressure (COP) values and multiple second COP values and calculates an accumulated difference corresponding to the candidate weight set according to the first COP values and the second COP values. The foot pressure measurement apparatus further determines a designated weight set from the candidate weight sets according to the accumulated differences.

### FIELD

The present invention relates to a foot pressure measurement apparatus, method, and non-transitory tangible machine-readable medium thereof. More particularly, the present invention relates to a foot pressure measurement apparatus, method, and non-transitory tangible machine-readable medium thereof that simulates a precise foot pressure by a smaller number of pressure sensors.

### BACKGROUND

Knowing foot pressure of a human being is extremely important in various fields. Taking medical science as an example, many diseases such as degenerative joint disease and plantar fasciitis are caused by abnormal foot pressure (e.g. unbalanced foot pressure). If a person can measure his or her foot pressure easily, the probabilities of having these diseases may be greatly reduced. Taking athletics as another example, an athlete needs information such as gait and foot pressure to improve his or her performance.

Nowadays, foot pressure measurement systems on the market can be classified into three categories, including medical insoles, optical three-dimensional measurement systems, and pressure sensing platforms. A medical insole can measure both static foot pressure and dynamic foot pressure and provide high precision measurement because a huge number of pressure sensors (e.g. 960 pressure sensors) are arranged therein. However, medical insoles have the drawbacks of being expensive, having short lifetimes, and being unable to be used for outdoor activities. Regarding optical three-dimensional measurement systems, only static foot pressure can be measured. Moreover, although center of foot pressure can be measured by an optical three-dimensional measurement system, the central line of foot pressure, which are useful in medical services, cannot be derived by accumulating several images. As to pressure sensing platforms, both static foot pressure and dynamic foot pressure can be measured. However, a pressure sensing platform has to be long enough (e.g. more than three meters) in order to measure dynamic foot pressure.

According to the above descriptions, it is clearly that none of the three existing categories of foot pressure measurement systems can be used in daily life. Therefore, foot pressure measurement systems that can be used in daily life is in an urgent need.

### SUMMARY

Provides is a foot pressure measurement apparatus. The foot pressure measurement apparatus can comprise a storage and a processor, wherein the processor is electrically connected to the storage. The storage stores a predetermined number of first pressure value sets corresponding to a first position set of a foot. The storage also stores the predetermined number of second pressure value sets corresponding to a second position set of the foot. A first position quantity of the first position set is smaller than a second position quantity of the second position set. The processor performs the following operations for a plurality of times: (a) deciding a candidate weight set, (b) calculating a plurality of first center of pressure (COP) values, wherein each of the first COP values is calculated according to the first position set, one of the first pressure value sets, and the candidate weight set, (c) calculating a plurality of second COP values, wherein each of the second COP values is calculated according to the second position set and one of the second pressure value sets, and (d) calculating an accumulated difference corresponding to the candidate weight set according to the first COP values and the second COP values. The processor further determines a designated weight set from the candidate weight sets according to the accumulated differences.

Also provided is a foot pressure measurement method, which is for use in an electronic computing apparatus. The electronic computing apparatus stores a predetermined number of first pressure value sets corresponding to a first position set of a foot. The electronic computing apparatus also stores the predetermined number of second pressure value sets corresponding to a second position set of the foot. A first position quantity of the first position set is smaller than a second position quantity of the second position set. The foot pressure measurement method performs the following steps for a plurality of times: (a) deciding a candidate weight set, (b) calculating a plurality of first COP values, wherein each of the first COP values is calculated according to the first position set, one of the first pressure value sets, and the candidate weight set, (c) calculating a plurality of second COP values, wherein each of the second COP values is calculated according to the second position set and one of the second pressure value sets, and (d) calculating an accumulated difference corresponding to the candidate weight set according to the first COP values and the second COP values. The foot pressure measurement method further comprises a step of determining a designated weight set from the candidate weight sets according to the accumulated differences.

Further provided is a non-transitory tangible machine-readable medium. The non-transitory tangible machine-readable medium stores a computer program comprising a plurality of codes. The codes perform a foot pressure measurement method when the computer program is loaded into an electronic computing apparatus. The electronic computing apparatus stores a predetermined number of first pressure value sets corresponding to a first position set of a foot. The electronic computing apparatus also stores the predetermined number of second pressure value sets corresponding to a second position set of the foot. A first position quantity of the first position set is smaller than a second position quantity of the second position set. The foot pressure measurement method performed by the codes is essentially the same as the one described in the preceding paragraph.

The foot pressure measurement technology herein (at least including apparatus, method, and non-transitory tangible machine-readable medium thereof) utilizes two kinds of pressure value sets, including the first pressure value sets corresponding to a first position set of a foot and the second pressure value sets corresponding to a second position set of the foot. The first pressure value sets are measured by a first insole that has a plurality of pressure sensors disposed at the positions indicated in the first position set, while the second pressure value sets are measured by a second insole that has a plurality of pressure sensors disposed at the positions indicated in the second position set. A first position quantity of the first position set is smaller than a second position quantity of the second position set.

The foot pressure measurement technology herein utilizes the first pressure value sets and the second pressure value sets to determine a designated weight set from a plurality of candidate weight set for simulating a COP value measured by the second insole (or an insole whose pressure sensors are disposed at the same positions or at similar positions as those of the second insole) by one or more pressure value set measured by the first insole (or an insole whose pressure sensors are disposed at the same positions or at similar positions as those of the first insole). Generally speaking, for each of the candidate weight sets, the foot pressure measurement technology of the present invention calculates a plurality of first COP values according to the first position set, the first pressure value sets, and the candidate weight set, calculates a plurality of second COP values according to the second position set and the second pressure value sets, and calculates an accumulated difference corresponding to the candidate weight set according to the first COP values and the second COP values. The foot pressure measurement technology of the present invention determines the designated weight set from the candidate weight sets according to the accumulated differences.

Thereafter, if a user wears the aforesaid first insole (or an insole whose pressure sensors are disposed at the same positions or at similar positions as those of the first insole), the COP value(s) of the user can be calculated based on the pressure value set(s) measured by the insole worn by the user, the first position set, and the designated weight set. The calculated COP value(s) of the user can be considered as precise as those calculated based on the pressure value set(s) measured by the second insole (or an insole whose pressure sensors are disposed at the same positions or at similar positions as those of the second insole). By the aforesaid operations, an insole can contain only few sensors and, therefore, can be used in our daily life.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1A illustrates a schematic view of the foot pressure measurement apparatus 1 of the first embodiment of the present invention;

FIG. 1B illustrates a concrete example regarding the nine non-overlapping areas A1, A2, A3, A4, A5, A6, A7, A8, A9 of a foot and the positions comprised in the position set PS1; and

FIG. 2 illustrates the flowchart of the foot pressure measurement method of the second embodiment of the present invention.

### DETAILED DESCRIPTION

In the following description, the foot pressure measurement apparatus, method, and non-transitory tangible machine-readable medium thereof provided by certain embodiments of the present invention will be explained with reference to example embodiments thereof. However, these example embodiments are not intended to limit the present invention to any environment, applications, examples, embodiments or implementations described in these example embodiments. Therefore, description of these example embodiments is only for purpose of illustration rather than to limit the scope of the present invention.

It shall be appreciated that, in the following embodiments and the attached drawings, elements unrelated to the present invention are omitted from depiction; and dimensions of elements and dimensional proportions among individual elements in the attached drawings are provided only illustration but not to limit the scope of the present invention.

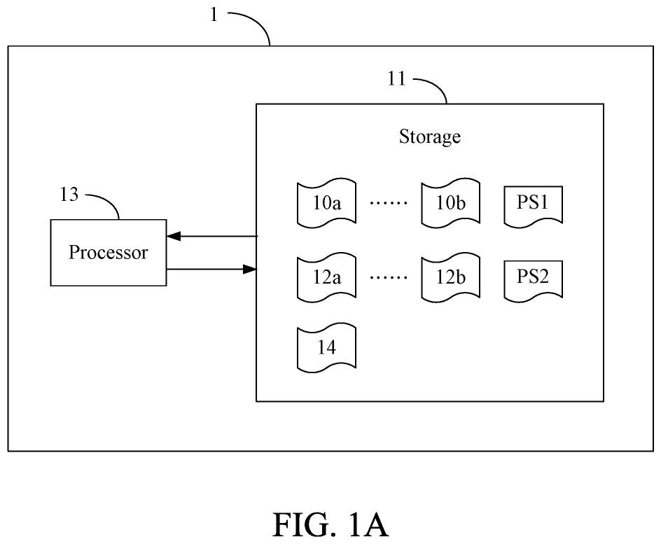

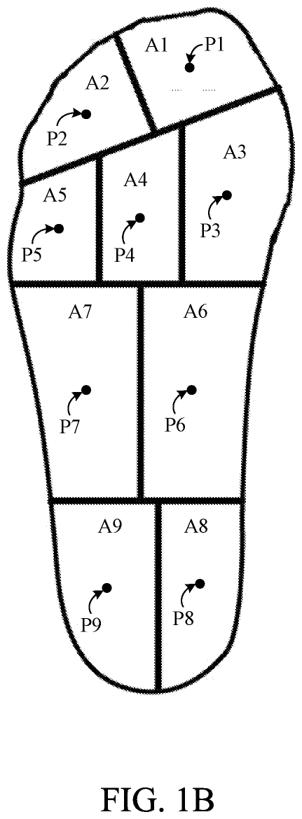

A first embodiment of the present invention is a foot pressure measurement apparatus 1 and a schematic view of which is illustrated in FIG. 1A. The foot pressure measurement apparatus 1 comprises a storage 11 and a processor 13, wherein the processor 13 is electrically connected to the storage 11. The storage 11 may be a memory, a hard disk drive (HDD), a universal serial bus (USB) disk, a compact disk (CD), or any other non-transitory storage medium or apparatus that is able to store digital data and well-known to those of ordinary skill in the art. The processor 13 may be one of various processing units, central processing units (CPUs), microprocessors, digital signal processors (DSPs), or any other computing apparatuses with the same function and well-known to those of ordinary skill in the art.

In this embodiment, the storage 11 stores a position set PS1 of a foot (not shown), wherein the position set PS1 comprises a plurality of positions of the foot. For better understanding, the concrete example shown in FIG. 1B will be referred to in the following descriptions, which, however, is not used to limit the scope of the present invention. In the example shown in FIG. 1B, a foot is divided into nine non-overlapping areas A1, A2, A3, A4, A5, A6, A7, A8, A9. The areas A1 and A2 correspond to the phalangeal bones of the foot, the areas A3, A4, A5 correspond to the metatarsal bones of the foot, the areas A6 and A7 correspond to the tarsal bones of the foot, and the areas A8 and A9 correspond to the heel bones of the foot.

The positions P1, P2, P3, P4, P5, P6, P7, P8, P9 are respectively designated for the areas A1, A2, A3, A4, A5, A6, A7, A8, A9. For example, the positions P1, P2, P3, P4, P5, P6, P7, P8, P9 may be the centers of the areas A1, A2, A3, A4, A5, A6, A7, A8, A9 respectively. The aforesaid position set PS1 comprises the positions P1, P2, P3, P4, P5, P6, P7, P8, P9. Please note that the present invention does not put restriction on the number of the areas that a foot can be divided into as long as the number is not smaller than three. In other words, the present invention does not put restriction on the position quantity of the position set PS1 as long as the position quantity is not smaller than three. In the case that the position quantity of the position set PS1 is three, the foot is divided into an upper area, a middle area, and a bottom area, wherein the upper area corresponds to the phalangeal bones of the foot, the middle area corresponds to the tarsal bones of the foot, and the bottom area corresponds to the heel bones of the foot.

A first insole is designed in a way that a first pressure sensor set (not shown) is placed in the first insole according to the position set PS1. To be more specific, a pressure sensor is placed at each of the positions P1, P2, P3, P4, P5, P6, P7, P8, P9 in the first insole, and these pressure sensors can be considered as the aforesaid first pressure sensor set. A foot wears a shoe having the first insole inside exercises according to a predetermined sequence of activity patterns. If the foot that wears the shoe having the first insole inside is a prosthetic foot, the prosthetic foot exercises according to the predetermined sequence of activity patterns under the control of a machine.

When the foot that wears the shoe having the first insole inside exercises according to the predetermined sequence of activity patterns, the first pressure sensor set periodically generates a pressure value set by sensing the pressure of the foot. Since the pressure sensors of the first pressure sensor set are placed at the positions P1, P2, P3, P4, P5, P6, P7, P8, P9 in the first insole, each pressure value set corresponds to the position set PS1. To be more specific, the sensed pressure values comprised in each pressure value set correspond to the positions P1, P2, P3, P4, P5, P6, P7, P8, P9 respectively.

In this embodiment, when the foot that wears the shoe having the first insole inside exercises according to the predetermined sequence of activity patterns, the first pressure sensor set generates a predetermined number (not shown) of pressure value sets 10a, . . . , 10b corresponding to the position set PS1 of the foot. The pressure value sets 10a, . . . , 10b corresponding to the position set PS1 of the foot are stored in the storage 11.

In this embodiment, a second insole is referred to as a simulation object. The second insole comprises a second pressure sensor set (not shown) inside. To be more specific, a plurality of pressure sensors are placed at a plurality of positions of the second insole respectively, wherein the pressure sensors form the second pressure sensor set and the positions form the position set PS2. Since the positions comprised in the position set PS2 corresponds to the second insole, they are considered as corresponding to the foot as well. The storage 11 also stores the position set PS2.

Please note that since the second insole is referred to as a simulation object, the number of the pressure sensors comprised in the first insole is smaller than the number of the pressure sensors comprised in the second insole. In other words, the position quantity of the position set PS1 is smaller than the position quantity of the position set PS2. To achieve a better simulation result, the second insole may be a medical insole that comprises a huge number of pressure sensors (e.g. 960 pressure sensors).

A foot wears a shoe having the second insole inside also exercises according to the aforesaid predetermined sequence of activity patterns. If the foot that wears the shoe having the second insole inside is a prosthetic foot, the prosthetic foot exercises according to the predetermined sequence of activity patterns under the control of a machine. When the foot that wears the shoe having the second insole inside exercises according to the predetermined sequence of activity patterns, the second pressure sensor set periodically generates a pressure value set by sensing the pressure of the foot. Since the second pressure sensor set are placed at the position set PS2 in the second insole, each pressure value set corresponds to the position set PS2.

In this embodiment, when the foot that wears the shoe having the second insole inside exercises according to the predetermined sequence of activity patterns, the second pressure sensor set generates a predetermined number of (not shown) of pressure value sets 12a, . . . , 12b corresponding to the position set PS2 of the foot. The pressure value sets 12a, . . . , 12b corresponding to the position set PS2 of the foot are stored in the storage 11.

In this embodiment, the processor 13 evaluates a plurality of candidate weight sets and then determines a designated weight set for simulating the second insole by the first insole. To be more specific, the processor 13 performs the following operations (a)-(d) for a plurality of times.

In the operation (a), the processor 13 determines a candidate weight set (not shown). The candidate weight set comprises a plurality of weight values, and the weight values comprised in the candidate weight set correspond to the positions P1, P2, P3, P4, P5, P6, P7, P8, P9 comprised in the position set PS1 one-on-one. It is noted that the candidate weight sets determined at different iterations are different. For the first iteration, the weight values comprised in the candidate weight set may be set to 1/N, wherein the variable N is equivalent to the candidate weight quantity of the candidate weight set (which is also equivalent to the position quantity of the position set PS1). In this embodiment, since the position quantity of the position set PS1 is 9, the weight values comprised in the candidate weight set may be all set to one-ninth in the first iteration.

In the operation (b), the processor 13 calculates a plurality of first center of pressure (COP) values. Specifically, for each of the pressure value sets 10a, . . . , 10b, the processor 13 calculates a first COP value according to the position set PS1, the pressure value set, and the candidate weight set.

In some embodiments, each of the first COP values comprises a first x-directional COP value and a first y-directional COP value. For those embodiments, the processor 13 may calculate each of the first COP values according to the following equation (1).

$$\{ \begin{matrix}{COP}_{x} = \frac{{\sum }_{i = 1}^{N} {z}_{i} \cdot {x}_{i} \cdot F ( {x}_{i} , {y}_{i} )}{{\sum }_{i = 1}^{N} {z}_{i} \cdot F ( {x}_{i} , {y}_{i} )} \\ {COP}_{y} = \frac{{\sum }_{i = 1}^{N} {z}_{i} \cdot {y}_{i} \cdot F ( {x}_{i} , {y}_{i} )}{{\sum }_{i = 1}^{N} {z}_{i} \cdot F ( {x}_{i} , {y}_{i} )}\end{matrix} \qquad ( 1 )$$

In the equation (1), the parameters COPₓ and COP_{y} respectively represent a first x-directional COP value and a first y-directional COP value of a first COP value, the variable zᵢ represents the i^{th} weight value comprised in the candidate weight set, the variables xᵢ and yᵢ respectively represent the x-directional coordinate and the y-directional coordinate of the i^{th} position in the position set PS1, the variable F(xᵢ, yᵢ) represents the i^{th} pressure value in the pressure value set, and the variable N represents the position quantity of the position set PS1 (which is equivalent to the candidate weight quantity of a candidate weight set and the pressure value quantity of each of the pressure value sets 10a, . . . , 10b). For the concrete example shown in FIG. 1B, the variable N is 9. As mentioned, the storage 11 stores the pressure value sets 10a, . . . , 10b. For each of the pressure value sets 10a, . . . , 10b, the processor 13 calculates a corresponding first COP value according to the above equation (1).

In the operation (c), the processor 13 calculates a plurality of second COP values. Specifically, for each of the pressure value sets 12a, . . . , 12b, the processor 13 calculates a second COP value according to the position set PS2 and the pressure value set.

In some embodiments, each of the second COP values comprises a second x-directional COP value and a second y-directional COP value. For those embodiments, the processor 13 may calculate each of the second COP values according to the following equation (2).

$$\{ \begin{matrix}{COP}_{x} = \frac{{\sum }_{i = 1}^{M} w \cdot {x}_{i} \cdot F ( {x}_{i} , {y}_{i} )}{{\sum }_{i = 1}^{M} w \cdot F ( {x}_{i} , {y}_{i} )} \\ {COP}_{y} = \frac{{\sum }_{i = 1}^{M} w \cdot {y}_{i} \cdot F ( {x}_{i} , {y}_{i} )}{{\sum }_{i = 1}^{M} w \cdot F ( {x}_{i} , {y}_{i} )}\end{matrix} \qquad ( 2 )$$

In the equation (2), the parameters COPₓ and COP_{y} respectively represent a second x-directional COP value and a second y-directional COP value of a second COP value, the variables xᵢ and yᵢ respectively represent the x-directional coordinate and the y-directional coordinate of the i^{th} position in the position set PS2, the variable F(xᵢ, yᵢ) represents the i^{th} pressure value in the pressure value set, and the variable M represents the position quantity of the position set PS2 (which is equivalent to the pressure value quantity of each of the pressure value sets 12a, . . . , 12b). In addition, the variable w represents a weight value and may be set to 1/M. As mentioned, the storage 11 stores the pressure value sets 12a, . . . , 12b. For each of the pressure value sets 12a, . . . , 12b, the processor 13 calculates a corresponding second COP value according to the above equation (2).

In the operation (d), the processor 13 calculates an accumulated difference corresponding to the candidate weight set according to the first COP values and the second COP values. In this embodiment, the number of the pressure value sets 10a, . . . , 10b and the number of the pressure value sets 12a, . . . , 12b are the same, which is the aforesaid predetermined number.

Since both the pressure value sets 10a, . . . , 10b and the pressure value sets 12a, . . . , 12b are derived when a foot exercises according to the predetermined sequence of activity patterns, the pressure value sets 10a, . . . , 10b correspond to the pressure value sets 12a, . . . , 12b one-on-one. Hence, in some embodiments, the processor 13 may calculates the accumulated difference corresponding to the candidate weight set by calculating a difference value between each of the first COP values and the corresponding second COP value and deriving the accumulated difference by summing up the differences. That is, the processor 13 may calculate the accumulated difference corresponding to the candidate weight set by the following equation:

$$O = {\sum }_{j = 1}^{S} \vert {COP}_{1 j} - {COP}_{2 j} \vert \qquad ( 3 )$$

In the equation (3), the parameter O represents the accumulated difference corresponding to the candidate weight, the parameter COP₁ⱼ represents the j^{th} first COP value, the parameter COP₂ⱼ represents the j^{th} second COP value, and the parameter S represents the predetermined number.

As mentioned, the processor 13 performs the above operations (a)-(d) for a plurality of times. For each time (i.e. each iteration), a candidate weight set is determined in the operation (a). In some embodiments, the weight values of the candidate weight sets that correspond to the same position are within a predetermined range. Moreover, in some embodiments, the processor 13 determines each of the candidate weight sets according to one of a differential evolution algorithm and a genetic algorithm.

In some embodiments, the processor 13 may determine whether a termination condition has met after each iteration of the aforesaid operations (a)-(d). For example, the processor 13 may determines whether a target number of iterations has reached. If the target number of iterations has reached, the processor 13 stops executing the operations (a)-(d). As another example, the processor 13 may determine whether the accumulated difference calculated in this iteration is smaller than a predetermined threshold. If the accumulated difference calculated in this iteration is smaller than the predetermined threshold, the processor 13 stops executing the operations (a)-(d).

After the processor 13 has performed the above operations (a)-(d) for a plurality of times, the processor 13 determines a designated weight set from the candidate weight sets according to the accumulated differences. For example, the processor 13 may select the candidate weight set with the smallest accumulated difference as the designated weight set. The designated weight set is used for simulating the second insole by the first insole.

In some embodiments, the storage 11 further stores a pressure value set 14 that corresponds to the position set PS1 and a user. It means that the pressure value set 14 is generated by the first pressure sensor set comprised in the first insole when the user wears a shoe having the first insole inside (or generated by the pressure senor set in another insole that is similar to the first insole when the user wear a shoe having that insole inside). For those embodiments, the processor 13 further calculates a third COP value for the user according to the position set PS1, the third pressure value set, and the designated weight set.

According to the above descriptions, the foot pressure measurement apparatus 1 utilizes the pressure value sets 10a, . . . , 10b and the pressure value sets 12a, . . . , 12b to determine a designated weight set for simulating the second insole by the first insole. If a user wears the aforesaid first insole (or an insole whose pressure sensors are disposed at the same positions or at similar positions as those of the first insole), the COP value(s) of the user can be calculated based on the pressure value set(s) measured by the insole worn by the user, the first position set, and the designated weight set. The calculated COP value(s) of the user can be considered as precise as those calculated based on the pressure value set(s) measured by the second insole (or an insole whose pressure sensors are disposed at the same positions or at similar positions as those of the second insole). By the aforesaid operations, an insole can contain only few sensors and, therefore, can be used in our daily life.

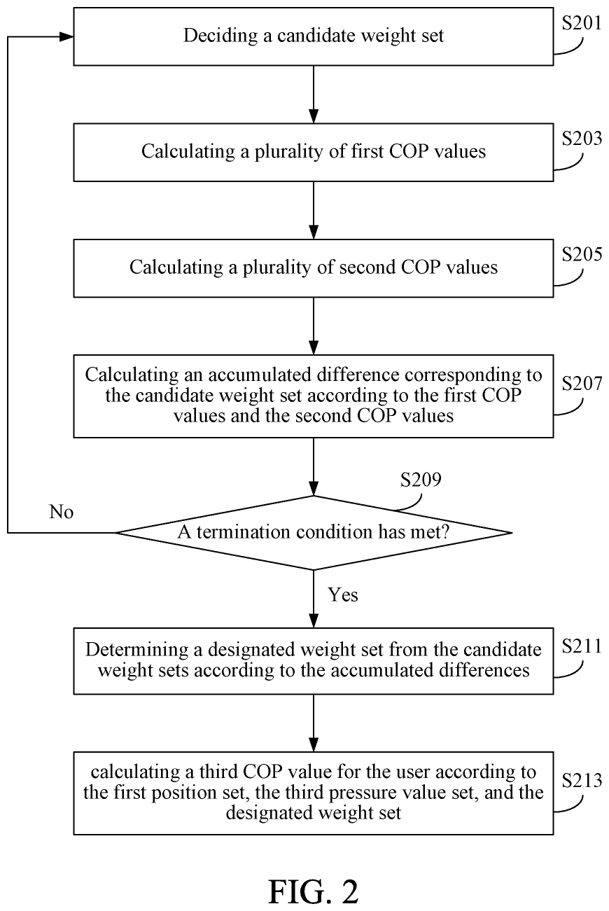

A second embodiment of the present invention is a foot pressure measurement method, whose flowchart is illustrated in FIG. 2. The foot pressure measurement method may be used in an electronic computing apparatus (e.g. the aforesaid foot pressure measurement apparatus 1). The electronic computing apparatus stores a predetermined number of first pressure value sets corresponding to a first position set of a foot. The electronic computing apparatus also stores the predetermined number of second pressure value sets corresponding to a second position set of the foot. The first position quantity of the first position set is smaller than a second position quantity of the second position set. In addition, the first position quantity is not smaller than three. In the case that the first position quantity of the first position set is three, the foot is divided into an upper area, a middle area, and a bottom area, wherein the upper area corresponds to the phalangeal bones of the foot, the middle area corresponds to the tarsal bones of the foot, and the bottom area corresponds to the heel bones of the foot.

In some embodiment, the predetermined number of first pressure value sets are sensed by a first pressure sensor set when a foot exercises according to a predetermine sequence of activity patterns, and the predetermined number of second pressure value sets are sensed by a second pressure sensor set when the foot exercises according to the predetermine sequence of activity patterns.

The foot pressure measurement method comprises steps S201 to S213. In the step S201, the electronic computing apparatus decides a candidate weight set. In the step S203, the electronic computing apparatus calculates a plurality of first COP values, wherein each of the first COP values is calculated according to the first position set, one of the first pressure value sets, and the candidate weight set. In the step S205, the electronic computing apparatus calculates a plurality of second COP values, wherein each of the second COP values is calculated according to the second position set and one of the second pressure value set.

In some embodiment, the first COP value calculated in the step S203 comprises a first x-directional COP value and a first y-directional COP value, and the second COP value calculated in the step S205 comprises a second x-directional COP value and a second y-directional COP value.

In the step S207, the electronic computing apparatus calculates an accumulated difference corresponding to the candidate weight set according to the first COP values and the second COP values. It is noted that the predetermined number of first pressure value sets correspond to the predetermined number of second pressure value sets one-on-one, and the first COP values correspond to the second COP values one-on-one. Hence, in some embodiments, the step S207 comprises a step of calculating a difference value between each of the first COP values and the corresponding second COP value the electronic computing apparatus and a step of deriving the accumulated difference by summing up the differences the electronic computing apparatus.

In the step S209, the electronic computing apparatus determine whether a termination condition has met. If the step S209 determines that the termination condition has not met, the foot pressure measurement method executes the aforesaid steps S201 to S209 again. If the step S209 determines that the termination condition has met, the foot pressure measurement method proceeds to the step S211. In the step S211, the electronic computing apparatus determines a designated weight set from the candidate weight sets according to the accumulated differences.

It is noted that the aforesaid steps S201 may be executed for a plurality of times. In some embodiments, the weight values of the candidate weight sets that correspond to the same position are within a predetermined range. Moreover, in some embodiments, the step S201 determines each of the candidate weight sets according to one of a differential evolution algorithm and a genetic algorithm.

In some embodiments, the electronic computing apparatus further stores a third pressure value set corresponding to the first position set, and the third sensed pressure value set corresponds to a user. For those embodiments, the foot pressure measurement method further executes the step S213 after the step S211. In step S213, the electronic computing apparatus calculates a third COP value for the user according to the first position set, the third pressure value set, and the designated weight set.

In addition to the aforesaid steps, the second embodiment can execute all the operations and steps of the foot pressure measurement apparatus 1 set forth in the first embodiment, have the same functions, and deliver the same technical effects as the first embodiment. How the second embodiment executes these operations and steps, has the same functions, and delivers the same technical effects as the first embodiment will be readily appreciated by those of ordinary skill in the art based on the explanation of the first embodiment and, thus, will not be further described herein.

It shall be appreciated that, in the specification and the claims of this patent application, some words (including insole, pressure sensor set, COP values) are preceded by the terms such as “first,” “second,” or “third.” Please note that the terms of “first,” “second,” and “third” are only used to distinguish these words.

The foot pressure measurement method described in the second embodiment may be implemented as a computer program having a plurality of codes. The computer program is stored in a non-transitory tangible machine-readable medium, which may be a read only memory (ROM), a flash memory, a floppy disk, a hard disk, a compact disk (CD), a digital versatile disc (DVD), a mobile disk, or any other storage media with the same function and well known to those of ordinary skill in the art. After the codes of the computer program loaded into an electronic computing apparatus (e.g., the foot pressure measurement apparatus 1), the computer program executes the foot pressure measurement method as described in the second embodiment.

According to the above descriptions, the foot pressure measurement technology of the present invention utilizes two kinds of pressure value sets to determine a designated weight set for simulating the second insole by the first insole. If a user wears the aforesaid first insole (or an insole whose pressure sensors are disposed at the same positions or at similar positions as those of the first insole), the COP value(s) of the user can be calculated based on the pressure value set(s) measured by the insole worn by the user, the first position set, and the designated weight set. The calculated COP value(s) of the user can be considered as precise as those calculated based on the pressure value set(s) measured by the second insole (or an insole whose pressure sensors are disposed at the same positions or at similar positions as those of the second insole). By the aforesaid operations, an insole can contain only few sensors and, therefore, can be used in our daily life.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A foot pressure measurement apparatus, comprising:
a storage, storing a predetermined number of first pressure value sets corresponding to a first position set of a foot and the predetermined number of second pressure value sets corresponding to a second position set of the foot, wherein a first position quantity of the first position set is smaller than a second position quantity of the second position set; and
a processor, being electrically connected to the storage and performing the following operations for a plurality of times:
(a) deciding a candidate weight set,
(b) calculating a plurality of first center of pressure (COP) values, wherein each of the first COP values is calculated according to the first position set, one of the first pressure value sets, and the candidate weight set,
(c) calculating a plurality of second COP values, wherein each of the second COP values is calculated according to the second position set and one of the second pressure value sets, and
(d) calculating an accumulated difference corresponding to the candidate weight set according to the first COP values and the second COP values,
wherein the processor further determines a designated weight set from the candidate weight sets according to the accumulated differences.

2. The foot pressure measurement apparatus of claim 1, wherein the storage further stores a third pressure value set corresponding to the first position set, the third sensed pressure value set corresponds to a user, and the processor further calculates a third COP value for the user according to the first position set, the third pressure value set, and the designated weight set.

3. The foot pressure measurement apparatus of claim 1, wherein each of the first COP values comprises a first x-directional COP value and a first y-directional COP value, and each of the second COP value comprises a second x-directional COP value and a second y-directional COP value.

4. The foot pressure measurement apparatus of claim 1, wherein the first position set comprises a plurality of positions, each of the candidate weight sets comprises a plurality of weight values, and the weight values comprised in each of the candidate weight sets correspond to the positions comprised in the first position set one-on-one.

5. The foot pressure measurement apparatus of claim 4, wherein the weight values of the candidate weight sets that correspond to the same position are within a predetermined range.

6. The foot pressure measurement apparatus of claim 1, wherein the processor determines each of the candidate weight sets according to one of a differential evolution algorithm and a genetic algorithm.

7. The foot pressure measurement apparatus of claim 1, wherein the first position quantity is not smaller than three.

8. The foot pressure measurement apparatus of claim 1, wherein the foot is divided into an upper area, a middle area, and a bottom area.

9. The foot pressure measurement apparatus of claim 1, wherein the predetermined number of first pressure value sets are sensed by a first pressure sensor set when a foot exercises according to a predetermined sequence of activity patterns, and the predetermined number of second pressure value sets are sensed by a second pressure sensor set when the foot exercises according to the predetermined sequence of activity patterns.

10. The foot pressure measurement apparatus of claim 1, wherein the predetermined number of first pressure value sets correspond to the predetermined number of second pressure value sets one-on-one, the first COP values correspond to the second COP values one-on-one, and the processor performs the operation (d) by calculating a difference value between each of the first COP values and the corresponding second COP value and deriving the accumulated difference by summing up the differences.

11. A foot pressure measurement method for use in an electronic computing apparatus, the electronic computing apparatus storing a predetermined number of first pressure value sets corresponding to a first position set of a foot and the predetermined number of second pressure value sets corresponding to a second position set of the foot, a first position quantity of the first position set being smaller than a second position quantity of the second position set, and the foot pressure measurement method comprising:
performing the following steps for a plurality of times:
(a) deciding a candidate weight set;
(b) calculating a plurality of first COP values, wherein each of the first COP values is calculated according to the first position set, one of the first pressure value sets, and the candidate weight set;
(c) calculating a plurality of second COP values, wherein each of the second COP values is calculated according to the second position set and one of the second pressure value sets; and
(d) calculating an accumulated difference corresponding to the candidate weight set according to the first COP values and the second COP values; and
determining a designated weight set from the candidate weight sets according to the accumulated differences.

12. The foot pressure measurement method of claim 11, wherein the electronic computing apparatus further stores a third pressure value set corresponding to the first position set, the third sensed pressure value set corresponds to a user, and the foot pressure method further comprises the following step of:
calculating a third COP value for the user according to the first position set, the third pressure value set, and the designated weight set.

13. The foot pressure measurement method of claim 11, wherein each of the first COP values comprises a first x-directional COP value and a first y-directional COP value, and each of the second COP value comprises a second x-directional COP value and a second y-directional COP value.

14. The foot pressure measurement method of claim 11, wherein the first position set comprises a plurality of positions, each of the candidate weight sets comprises a plurality of weight values, and the weight values comprised in each of the candidate weight sets correspond to the positions comprised in the first position set one-on-one.

15. The foot pressure measurement method of claim 14, wherein the weight values of the candidate weight sets that correspond to the same position are within a predetermined range.

16. The foot pressure measurement method of claim 11, wherein the step (a) determines the candidate weight set according to one of a differential evolution algorithm and a genetic algorithm.

17. The foot pressure measurement method of claim 11, wherein the first position quantity is not smaller than three.

18. The foot pressure measurement method of claim 11, wherein the foot is divided into an upper area, a middle area, and a bottom area.

19. The foot pressure measurement method of claim 11, wherein the predetermined number of first pressure value sets are sensed by a first pressure sensor set when a foot exercises according to a predetermined sequence of activity patterns, and the predetermined number of second pressure value sets are sensed by a second pressure sensor set when the foot exercises according to the predetermined sequence of activity patterns.

20. The foot pressure measurement method of claim 11, wherein the predetermined number of first pressure value sets correspond to the predetermined number of second pressure value sets one-on-one, the first COP values correspond to the second COP values one-on-one, and the step (d) comprises a step of calculating a difference value between each of the first COP values and the corresponding second COP value and a step of deriving the accumulated difference by summing up the differences.

21. A non-transitory tangible machine-readable medium, storing a computer program comprising a plurality of codes, the codes being able to perform a foot pressure measurement method when the computer program is loaded into an electronic computing apparatus, the electronic computing apparatus storing a predetermined number of first pressure value sets corresponding to a first position set of a foot and the predetermined number of second pressure value sets corresponding to a second position set of the foot, a first position quantity of the first position set being smaller than a second position quantity of the second position set, and the foot pressure measurement method comprising:
performing the following steps for a plurality of times:
deciding a candidate weight set;
calculating a plurality of first COP values, wherein each of the first COP values is calculated according to the first position set, one of the first pressure value sets, and the candidate weight set;
calculating a plurality of second COP values, wherein each of the second COP values is calculated according to the second position set and one of the second pressure value sets; and
calculating an accumulated difference corresponding to the candidate weight set according to the first COP values and the second COP values; and
determining a designated weight set from the candidate weight sets according to the accumulated differences.
